(19) United States
(12) Patent Application Publication
(10) Pub. No.: US 2017/0165455 A1
(43) Pub. Date: Jun. 15, 2017

(54) MEDICAL SYSTEM

(71) Applicant: OLYMPUS CORPORATION, Tokyo (JP)

(72) Inventor: Kazutoshi KUMAGAI, Tokyo (JP)

(73) Assignee: OLYMPUS CORPORATION, Tokyo (JP)

(21) Appl. No.: 15/446,403

(22) Filed: Mar. 1, 2017

Related U.S. Application Data
(63) Continuation of application No. PCT/JP2014/077065, filed on Oct. 9, 2014.

(51) Int. Cl. A61M 25/01; A61M 25/06; A61B 17/29

(52) U.S. Cl. CPC A61M 25/0127; A61M 25/0147; A61B 17/29; A61M 25/0662; A61M 2210/122

(57) ABSTRACT

A medical system including: a plurality of medical instruments each including an elongated insertion section that can be inserted into the body from the body surface; fixing parts that are provided in the insertion sections of the plurality of medical instruments, respectively, at positions away from the distal ends toward the proximal end side and that can be coupled with or separated from each other; and manipulation parts that are provided at the proximal end side of the insertion sections and that are used to manipulate coupling and separation of the fixing parts.

### CROSS-REFERENCE TO RELATED APPLICATIONS

This is a continuation of International Application PCT/JP2014/077065, with an international filing date of Oct. 9, 2014, which is hereby incorporated by reference herein in its entirety. This application claims the benefit of International Application PCT/JP2014/077065.

### TECHNICAL FIELD

The present invention relates to medical systems.

### BACKGROUND ART

In a known operation method for observing and treating the heart in the related art, a sheath is inserted into the pericardial cavity between the heart and the pericardium from the outside of the body, and a medical instrument, such as an endoscope or a treatment instrument, is led into the pericardial cavity via a route ensured inside the sheath (for example, see PTL 1).

### CITATION LIST

### Patent Literature

{PTL 1} Japanese Translation of PCT International Application, Publication No. 2010-535537

### SUMMARY OF INVENTION

### Technical Problem

An object of the present invention is to provide a medical system with which a plurality of medical instruments can be simultaneously and easily manipulated even in a place like the pericardial cavity, where the medical instruments directly receive body movements.

### Solution to Problem

An aspect of the present invention provides a medical system including: a plurality of medical instruments each having an elongated insertion section that can be inserted into the body from the body surface; fixing parts that are provided on the insertion sections of the plurality of medical instruments, at positions away from the distal ends toward the proximal end side, and that can be coupled with and separated from each other; and manipulation parts that are provided at the proximal end side of the insertion sections of the plurality of medical instruments and that manipulate coupling and separation of the fixing parts.

In the above aspect, the fixing parts may include magnetic bodies generating magnetic forces that attract each other.

In the above aspect, the magnetic bodies may be electromagnets.

In the above aspect, at least one of the medical instruments may include a flexible bending section provided closer to the distal-end side than the fixing part is.

In the above aspect, the medical system may further include a position changing mechanism that changes the positions of the fixing parts in the longitudinal direction of the insertion sections.

In the above aspect, the medical system may further include an anti-rotation mechanism that prevents the plurality of insertion sections coupled with each other at the fixing parts from relatively rotating about an axis extending in a direction passing through the fixing parts and intersecting the direction in which the plurality of insertion sections are arranged.

In the above aspect, the anti-rotation mechanism may include the plurality fixing parts provided on each of the plurality of insertion sections, at positions away from each other in the longitudinal direction. Alternatively, the anti-rotation mechanisms may include steps that are formed at contact surfaces of the fixing parts and that are engaged with each other in the radial direction of the insertion sections.

In the above aspect, the medical instrument may be an endoscope, a sheath, or an introducer.

### BRIEF DESCRIPTION OF DRAWINGS

FIG. 1 is a diagram showing the overall configuration of a medical system according to an embodiment of the present invention.

FIG. 2 is a diagram for explaining a method for using two sheaths in FIG. 1.

FIG. 3 is a diagram for explaining another method for using the two sheaths in FIG. 1.

FIG. 4 is a diagram for explaining a method for separating coupled magnets by using an over tube.

FIG. 5 is a diagram showing a modification of the fixing parts in FIG. 1.

FIG. 6 is a diagram showing another modification of the fixing parts in FIG. 1.

FIG. 7 is a diagram showing another modification of the fixing parts in FIG. 1.

FIG. 8 is a diagram showing another modification of the fixing parts in FIG. 1.

FIG. 9 is a diagram showing another modification of the fixing parts in FIG. 1.

FIG. 10 is a diagram showing another modification of the fixing parts in FIG. 1.

FIG. 11 is a diagram showing another modification of the fixing parts in FIG. 1.

FIG. 12A is a diagram showing another modification of the fixing parts in FIG. 1.

FIG. 12B is a diagram showing a state in which steps of the fixing parts in FIG. 12A are engaged with each other.

FIG. 13 is a diagram showing another modification of the fixing parts in FIG. 1.

FIG. 14 is a diagram showing another modification of the fixing parts in FIG. 1.

FIG. 15 is a diagram for explaining relative rotation of distal end portions of insertion sections that are each provided with one fixing part.

### DESCRIPTION OF EMBODIMENTS

A medical system 100 according to an embodiment of the present invention will be described below with reference to the drawings.

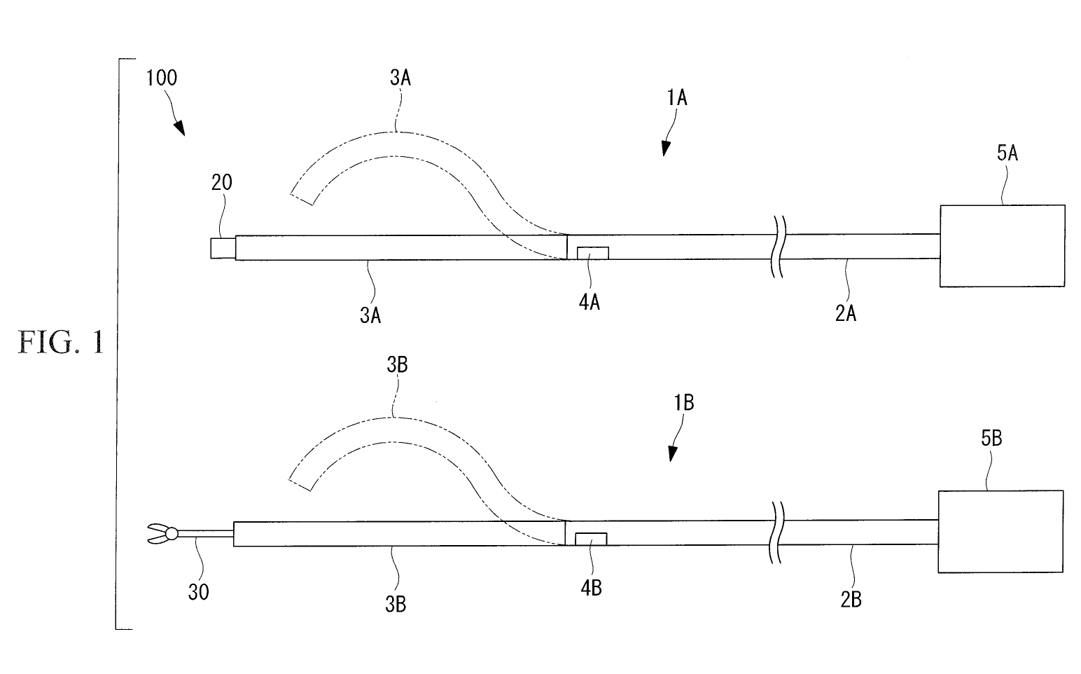

As shown in FIG. 1, the system 100 according to this embodiment includes two sheaths (medical instruments) 1A and 1B.

The sheaths 1A and 1B respectively include elongated cylindrical insertion sections 2A and 2B that can be inserted into the body, bending sections 3A and 3B and fixing parts 4A and 4B provided in the insertion sections 2A and 2B, and manipulation parts 5A and 5B connected to the proximal ends of the insertion sections 2A and 2B.

The insertion sections 2A and 2B have longitudinally penetrating lumens 2a and 2b, through which an endoscope 20, a treatment instrument 30, or the like are inserted. The insertion sections 2A and 2B are made of, for example, plastic, and they have such rigidity that they can transmit, to the distal ends, longitudinal linear motion and twisting motion about the longitudinal axis applied to the manipulation parts 5A and 5B and such flexibility that they can be bent in accordance with the shapes of the tissues in the living body.

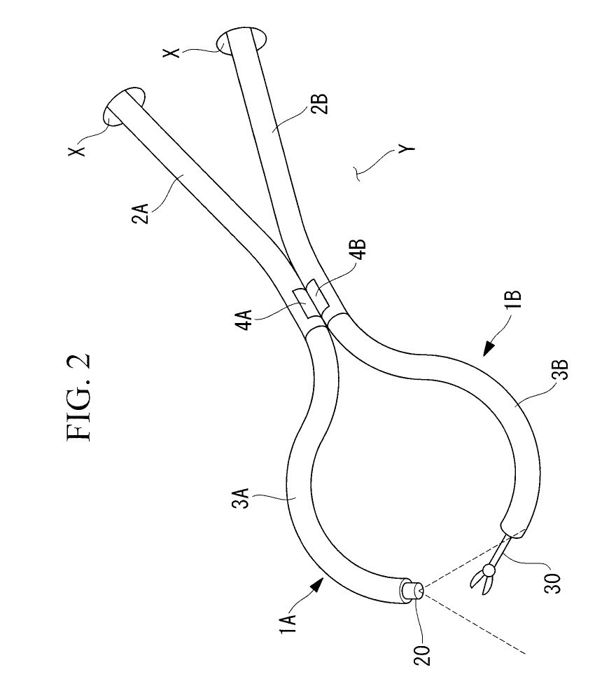

The bending sections 3A and 3B are provided at the distal end portions of the insertion sections 2A and 2B and are configured such that they can be bent as a result of an operator manipulating an angle knob or the like (not shown) provided on the manipulation parts 5A and 5B. As shown in FIG. 1, the respective bending sections 3A and 3B can be bent in a substantially semicircular arc shape projecting outward in the radial direction of the insertion sections 2A and 2B. Hence, as shown in FIG. 2, when the bending sections 3A and 3B are bent so as to project to opposite sides from each other with the insertion sections 2A and 2B being arranged substantially side-by-side, the distal ends of the insertion sections 2A and 2B substantially face each other.

The fixing parts 4A and 4B are formed of permanent magnets (hereinbelow, also referred to as magnets 4A and 4B) fixed to the outer circumferential surfaces of the insertion sections 2A and 2B and are provided on closer to the proximal-end side than the bending sections 3A and 3B, near the bending sections 3A and 3B. The magnet 4A provided on one insertion section 2A and the magnet 4B provided on the other insertion section 2B have opposite magnetic polarities and generate magnetic forces that attract each other.

The magnetic forces of the magnets 4A and 4B are designed such that the magnetic forces acting on the magnets 4A and 4B in a state in which the magnets 4A and 4B are coupled together are substantially equal to or smaller than the flexural rigidity and the torsional rigidity of the insertion sections 2A and 2B. This enables an operator to separate the magnets 4A and 4B that are coupled together by magnetic force, by pushing or pulling to advance or retract one of the two insertion sections 2A and 2B in the longitudinal direction or by rotating it about the longitudinal axis, while keeping the position of the other fixed.

Here, as shown in FIG. 2, the magnets 4A and 4B are provided only at portions of the insertion sections 2A and 2B in the circumferential direction, at the side opposite to the side on which the bending sections 3A and 3B are bent in a projecting shape. Hence, when the magnets 4A and 4B are coupled together, the insertion sections 2A and 2B are arranged side-by-side in such phases (rotation angles about the longitudinal axis) that the bending shapes of the bending sections 3A and 3B project toward the opposite sides, as shown in FIG. 2.

Next, the operation of the thus-configured medical system 100 will be described.

When treatment inside the pericardial cavity Y is performed by using the medical system 100 according to this embodiment, first, an operator separately inserts the insertion sections 2A and 2B of the two sheaths 1A and 1B into the body from below the xiphisternum, and portions of the insertion sections 2A and 2B on the distal end side are disposed inside the pericardial cavity Y through holes X provided in the pericardium. Next, the operator manipulates the manipulation parts 5A and 5B located outside the body to move the insertion sections 2A and 2B, such that the magnets 4A and 4B approach each other while facing each other. When the magnets 4A and 4B have reached positions a certain distance from each other, the magnets 4A and 4B spontaneously approach each other and are coupled together by the magnetic force.

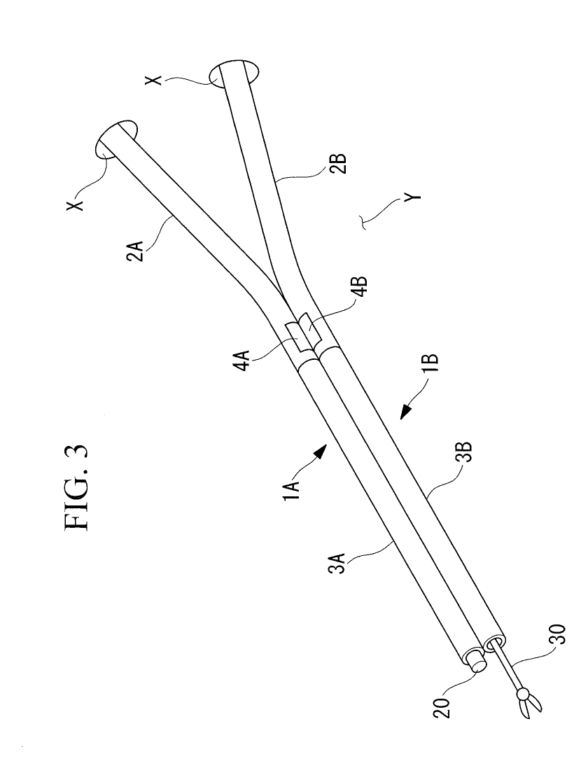

In a state in which the insertion sections 2A and 2B are coupled together with the magnets 4A and 4B and extend substantially parallel to each other, when, as shown in FIG. 3, an endoscope 20 is inserted into one insertion section 2A, and a treatment instrument 30 is inserted into the other insertion section 2B, it is possible to observe the treatment instrument 30 projecting from the distal end of the other insertion section 2B from behind with the endoscope 20 projecting from the distal end of the one insertion section 2A. As shown in FIG. 2, when the bending sections 3A and 3B are bent to make the distal ends of the insertion sections 2A and 2B substantially face each other, it is possible to observe the treatment instrument 30 from the substantially front side with the endoscope 20.

When removing the insertion sections 2A and 2B from the body, the operator separates the coupled magnets 4A and 4B by pushing, pulling, or twisting the manipulation part 5A or 5B corresponding to one of the insertion sections 2A and 2B, while maintaining the position of the other of them. As a result, the two insertion sections 2A and 2B become independently manipulatable, and thus, the two insertion sections 2A and 2B can be separately extracted from the pericardial cavity Y.

Here, according to this embodiment, in a state in which the insertion sections 2A and 2B are coupled together with the magnets 4A and 4B, as shown in FIG. 2 and FIG. 3, the relative positions of the distal end portions of the two insertion sections 2A and 2B, which are closer to the distal-end side than the magnets 4A and 4B, are maintained substantially constant. Accordingly, the operator can integrally manipulate the distal end portions of the two insertion sections 2A and 2B. For example, the operator can move the distal end portions of the two insertion sections 2A and 2B together in the front-rear directions by pushing or pulling the proximal end portion of one of the insertion sections 2A and 2B and can rotate the distal end portions of the two insertion sections 2A and 2B together by twisting the proximal end portion of one of the insertion sections 2A and 2B.

This leads to an advantage in that it is possible to easily manipulate the distal end portions of the two insertion sections 2A and 2B at will and dispose them at desired positions inside the pericardial cavity Y, even in a situation in which the insertion sections 2A and 2B inside the pericardial cavity Y directly receive the pulsation of the heart adjacent thereto and are moved. Another advantage is that, because the relative position between the endoscope 20 and the treatment instrument 30 is stably maintained even if the endoscope 20 and the treatment instrument 30 are moved by pulsation, it is possible to easily perform treatment with the treatment instrument 30, while continuing stable observation of the treatment instrument 30 with the endoscope 20.

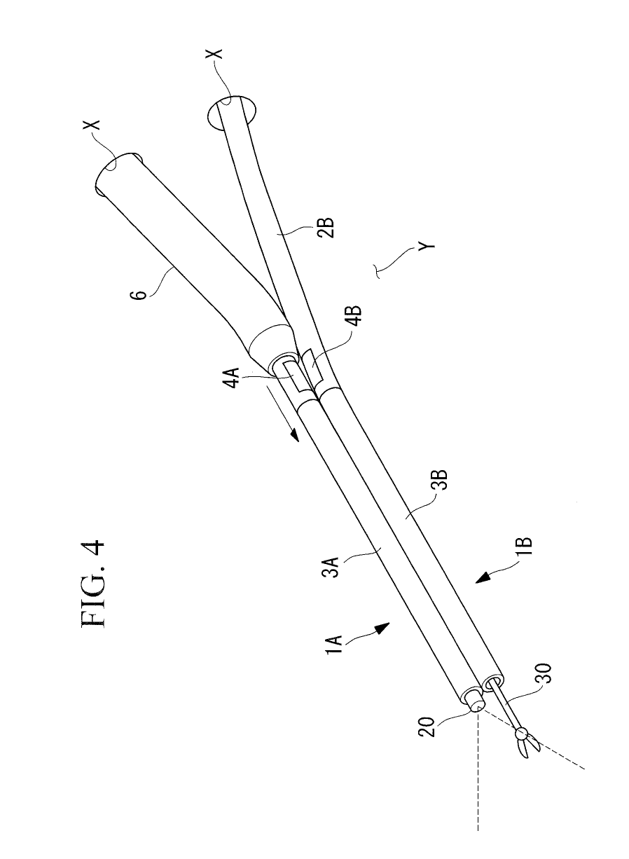

In this embodiment, an over tube 6 formed of a non-magnetic body, as shown in FIG. 4, may be used to separate the coupled magnets 4A and 4B. The over tube 6 has an inside diameter greater than the outside diameter of the insertion sections 2A and 2B and is attachable to the exterior of the insertion section 2A or 2B in such a manner that the insertion section 2A or 2B is inserted therethrough.

The coupled magnets 4A and 4B can be easily and reliably separated by inserting the over tube 6 attached to the exterior of one insertion section 2A into the pericardial cavity Y along the insertion section 2A, and advancing the distal end of the over tube 6 closer to the distal-end side than the magnets 4A and 4B, while forcing the distal end of the over tube 6 between the coupled magnets 4A and 4B. In a state in which the magnet 4A or 4B is covered by the over tube 6, the over tube 6 inhibits the magnets 4A and 4B from coupling together. Thus, the respective insertion sections 2A and 2B can be independently manipulated. It is desirable that the distal end portion of the over tube 6 be formed in a tapered shape gradually narrowing toward the distal end, to reduce the contact resistance with the peripheral tissues when moving in the living body.

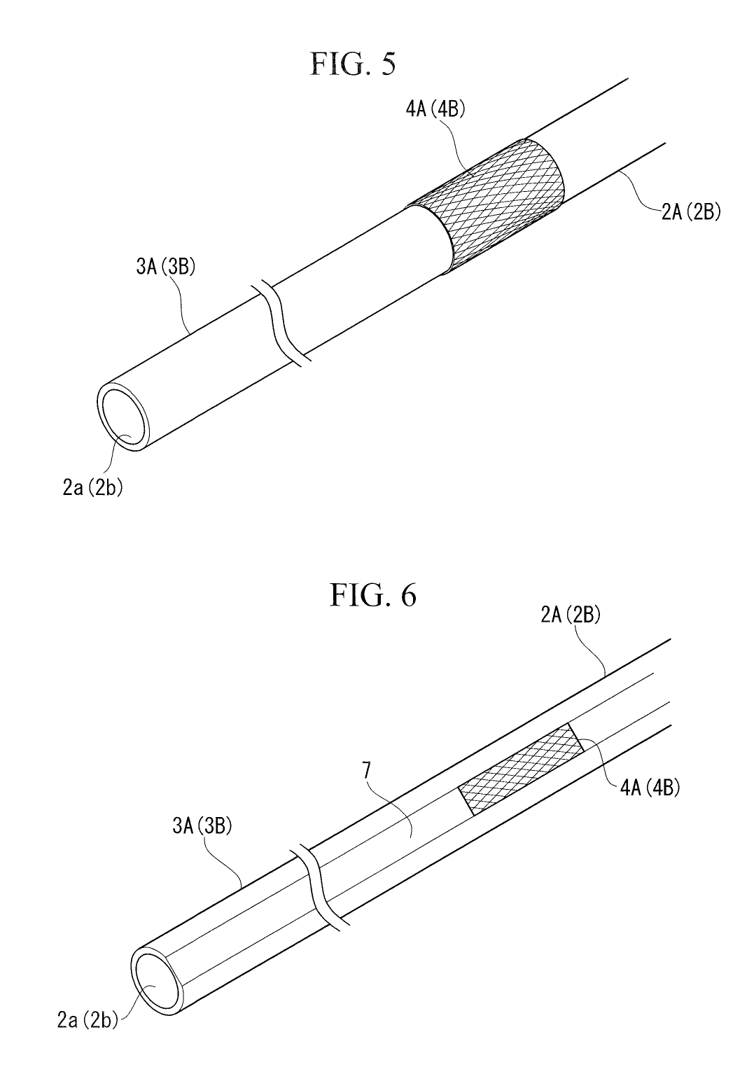

In this embodiment, although the magnets 4A and 4B are provided only at portions of the insertion sections 2A and 2B in the circumferential direction, instead, as shown in FIG. 5, they may be provided over the entire circumferences of the insertion sections 2A and 2B in the circumferential direction.

With this configuration, the magnets 4A and 4B are coupled together, regardless of the relative phases of the two insertion sections 2A and 2B. Accordingly, there is no need to adjust the phases of the two insertion sections 2A and 2B when the magnets 4A and 4B are coupled together inside the pericardial cavity Y, and the magnets 4A and 4B can be coupled together simply by bringing the magnets 4A and 4B toward each other. Because it is possible to rotate only one of the insertion sections 2A and 2B about the longitudinal axis while maintaining the magnets 4A and 4B in a coupled state, it is possible to change the phases of the distal end portions of the insertion sections 2A and 2B independently of each other while maintaining the relative positions of the distal end portions of the insertion sections 2A and 2B substantially constant and, thus, to change the orientations of the distal ends of the two insertion sections 2A and 2B independently of each other.

In this embodiment, although the magnets 4A and 4B that are curved in accordance with the curved shape of the outer circumferential surfaces of the insertion sections 2A and 2B are provided, instead, as shown in FIG. 6, it is possible to provide flat surfaces 7 at portions of the outer circumferential surfaces of the insertion sections 2A and 2B and to provide flat magnets 4A and 4B on the flat surfaces 7.

This makes it possible to provide larger contact areas between the magnets 4A and 4B and, thus, to increase the coupling force of the magnets 4A and 4B.

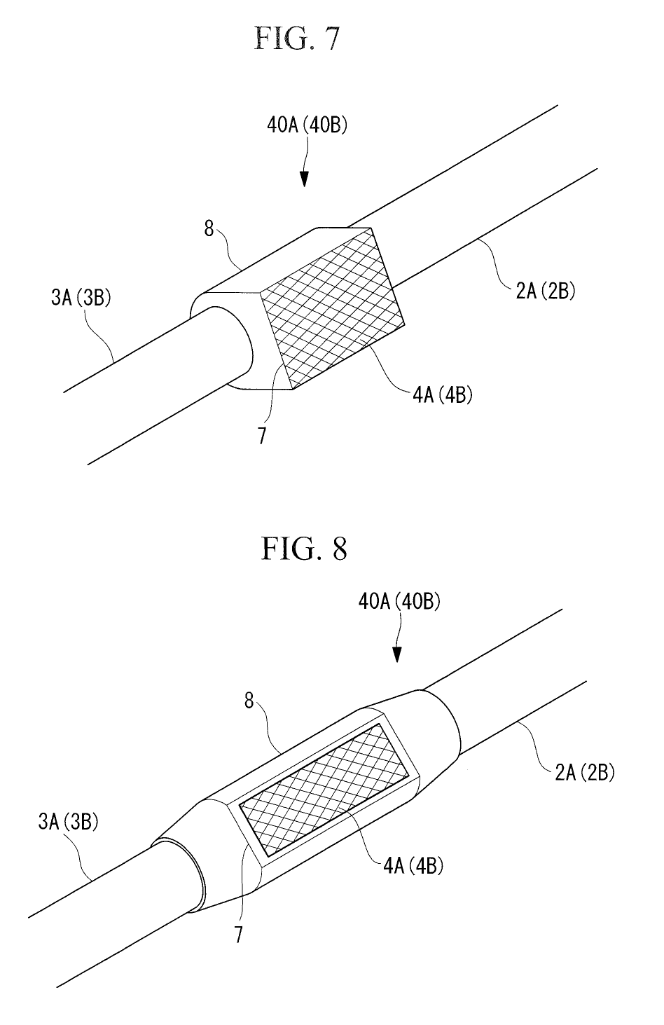

In this embodiment, although the fixing parts 4A and 4B that are formed of permanent magnets and are fixed to the insertion sections 2A and 2B have been described, instead, as shown in FIG. 7 to FIG. 10, fixing parts 40A and 40B may be attachments that are attached to the outer circumferential surfaces of the insertion sections 2A and 2B.

The fixing parts 40A and 40B shown in FIG. 7 and FIG. 8 include tubular tube members 8 attached to the outer circumferential surfaces of the insertion sections 2A and 2B, and the magnets 4A and 4B fixed to the outer circumferential surfaces of the tube members 8. It is desirable that the magnets 4A and 4B here be flat magnets that are fixed to the flat surfaces 7 formed on the outer circumferential surfaces of the tube members 8, similarly to the magnets 4A and 4B in FIG. 6.

The tube members 8 may be fixed to the outer circumferential surfaces of the insertion sections 2A and 2B.

Alternatively, the tube members (position changing mechanisms) 8 may be slidable in the longitudinal direction of the insertion sections 2A and 2B, while they may be temporarily fixed to the insertion sections 2A and 2B by the friction between the inner circumferential surfaces of the tube members 8 and the outer circumferential surfaces of the insertion sections 2A and 2B. This enables the positions of the fixing parts 40A and 40B to be easily changed in the longitudinal direction of the insertion sections 2A and 2B according to the attaching positions of the tube members 8 to the insertion sections 2A and 2B.

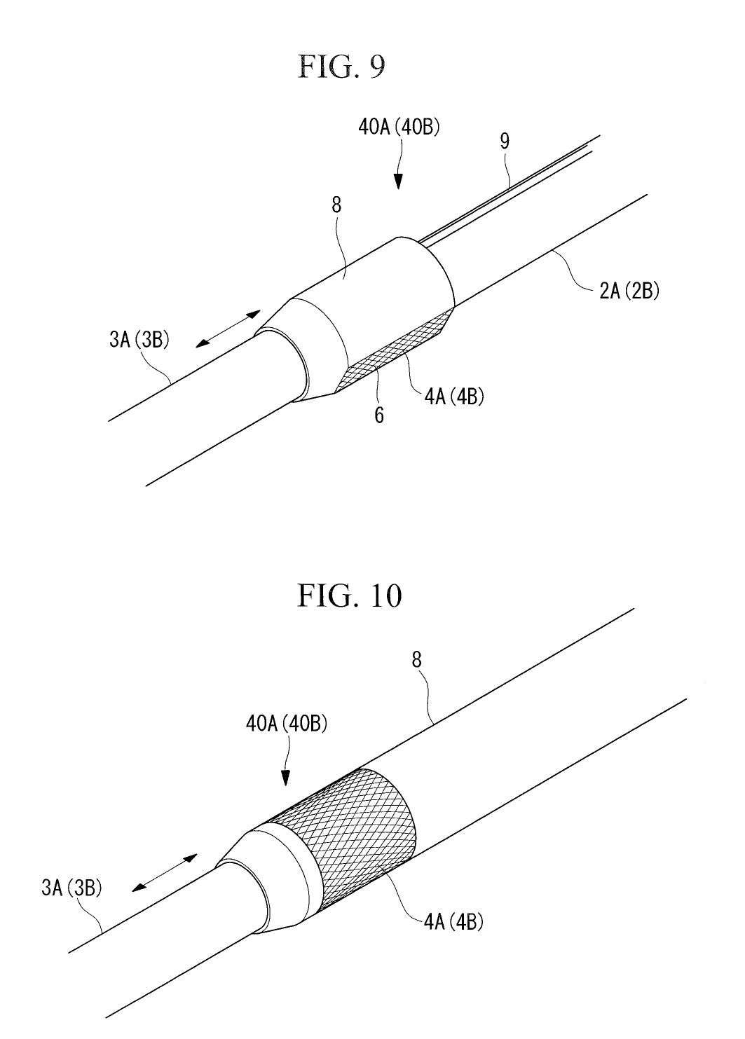

In the fixing parts 40A and 40B shown in FIG. 9, manipulation wires (position changing mechanisms) 9 extending to the proximal end side of the insertion sections 2A and 2B in the longitudinal direction of the insertion sections 2A and 2B are connected to the proximal ends of the tube members 8, which are slidable in the longitudinal direction, along the outer circumferential surfaces of the insertion sections 2A and 2B. The operator can move the fixing parts 40A and 40B disposed inside the pericardial cavity Y to arbitrary positions in the longitudinal direction, relative to the insertion sections 2A and 2B, by pushing or pulling the proximal end portions of the manipulation wires 9 located outside the body.

The fixing parts 40A and 40B shown in FIG. 10 include long, tube-shaped tube members (position changing mechanisms) 8 that accommodate portions of the insertion sections 2A and 2B that are closer to the proximal-end side than the bending sections 3A and 3B in a manner allowing them to move in the longitudinal direction, and magnets 4A and 4B fixed to the outer circumferential surfaces of the distal end portions of the tube members 8. The operator can move the fixing parts 40A and 40B disposed inside the pericardial cavity Y to arbitrary positions in the longitudinal direction, relative to the insertion sections 2A and 2B, by pushing or pulling the proximal ends of the tube members 8 located outside the body relative to the insertion sections 2A and 2B, thereby advancing or retracting the tube members 8 relative to the insertion sections 2A and 2B.

The fixing parts 40A and 40B shown in FIG. 9 and FIG. 10 may be attached to the insertion sections 2A and 2B before the insertion sections 2A and 2B are inserted into the body, or they may be attached to appropriate positions in the insertion sections 2A and 2B after the insertion sections 2A and 2B are inserted into the pericardial cavity Y, by advancing them along the insertion sections 2A and 2B.

With the fixing parts 40A and 40B shown in FIG. 9 and FIG. 10, the coupled magnets 4A and 4B can be easily and reliably separated without using the above-described over tube 6, by moving only one of the two fixing parts 40A and 40B in the longitudinal direction.

In this embodiment, fixing parts (anti-rotation mechanisms) may be provided in the insertion sections 2A and 2B, at two or more positions away from each other in the longitudinal direction.

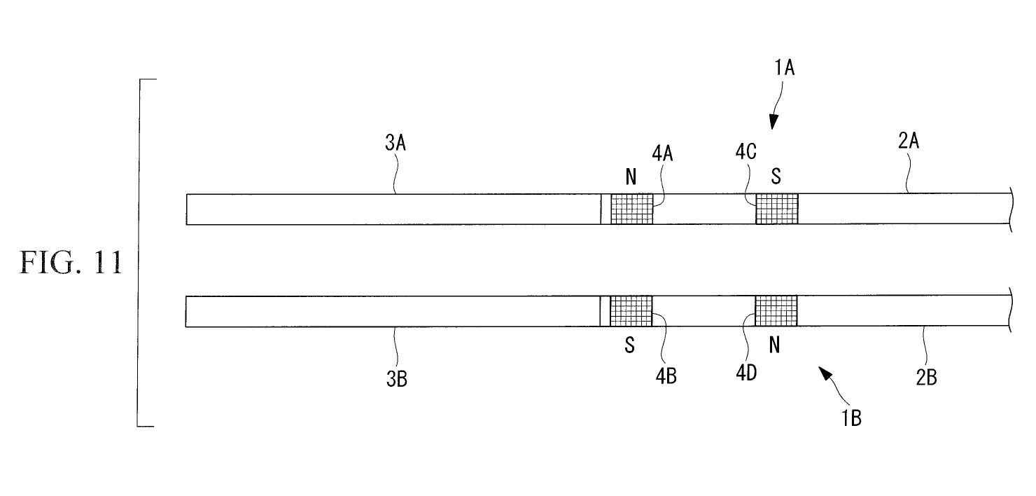

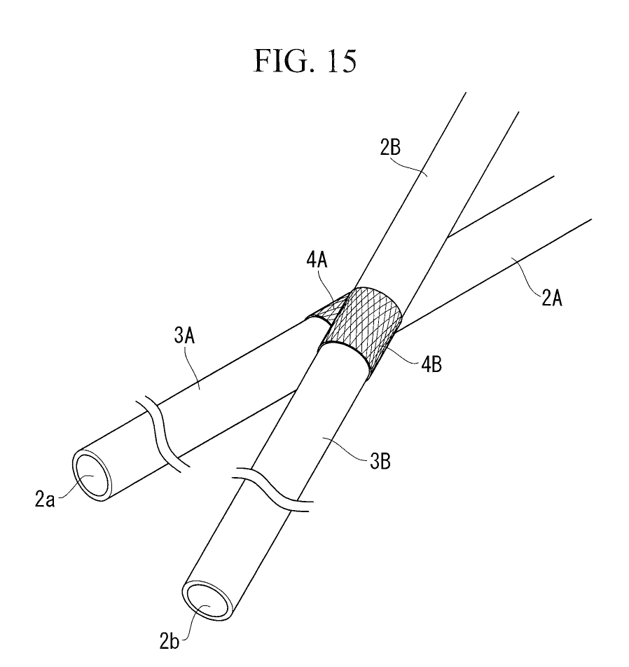

As shown in FIG. 15, when the fixing parts 4A and 4B are provided only at one position in the insertion sections 2A or 2B, the distal end portions of the insertion sections 2A and 2B could relatively rotate about an axis extending in the direction in which the insertion sections 2A and 2B are arranged, so as to pivot about the fixing parts 4A and 4B. Hence, as shown in FIG. 11, by providing fixing parts 4A and 4C; 4B and 4D in the insertion sections 2A and 2B, at two or more positions away from each other in the longitudinal direction, and by coupling the two insertion sections 2A and 2B at the two or more positions away from each other in the longitudinal direction, the distal end portions of the insertion sections 2A and 2B can be maintained parallel to each other.

At this time, it is desirable that the N-pole magnet 4A and the S-pole magnet 4C be alternately arranged in this order from the distal end side in one insertion section 2A and that the S-pole magnet 4B and the N-pole magnet 4D be alternately arranged in this order from the distal end side in the other insertion section 2B. With this configuration, even though a plurality of magnets 4A and 4C; 4B and 4D are provided in the insertion sections 2A and 2B, respectively, because the coupling positions of the magnets 4A and 4B; 4C and 4D are restricted by the magnetic polarity, it is possible to accurately manipulate the coupling positions of the insertion sections 2A and 2B.

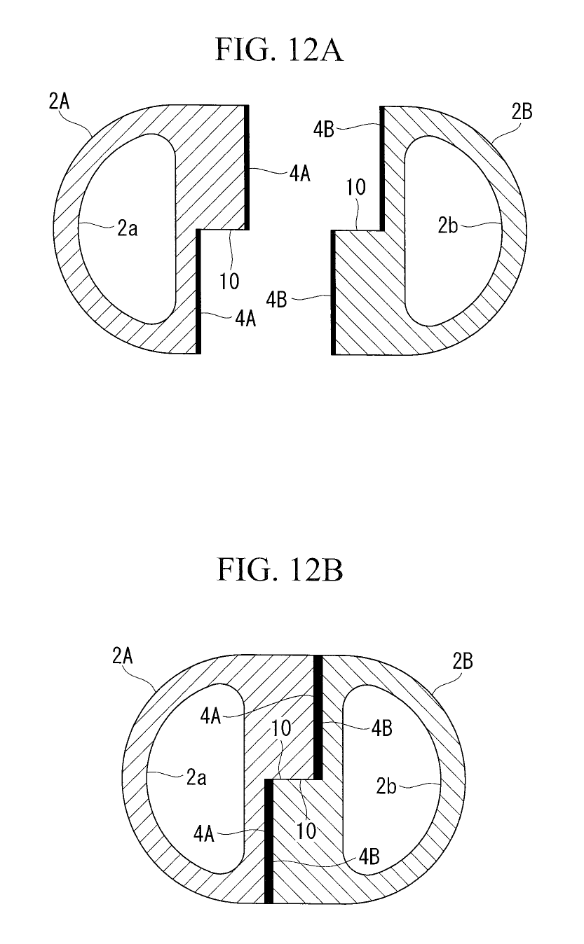

In this embodiment, as shown in FIG. 12A, steps (anti-rotation mechanisms) 10 that are engaged with each other in the radial direction of the insertion sections 2A and 2B may be provided on contact surfaces of the fixing parts 4A and 4B.

Also with this configuration, when moments about the fixing parts 4A and 4B act on the distal end portions of the insertion sections 2A and 2B, as shown in FIG. 12B, it is possible to inhibit relative rotation of the insertion sections 2A and 2B with the steps 10 that are engaged with each other in the direction intersecting the direction of the relative rotation.

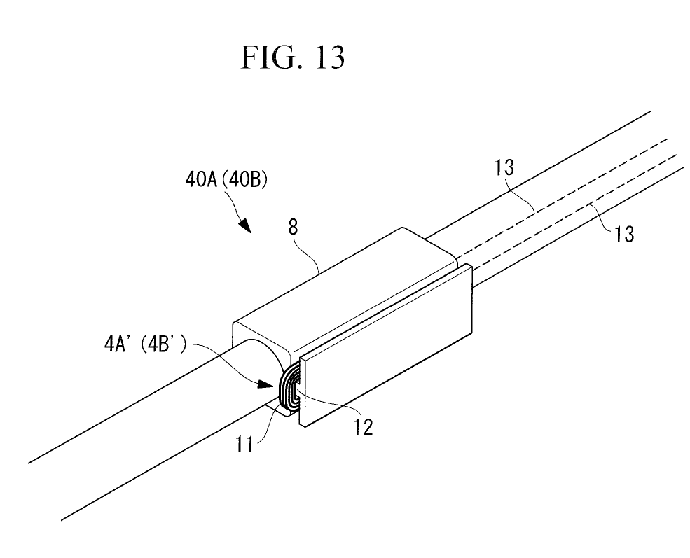

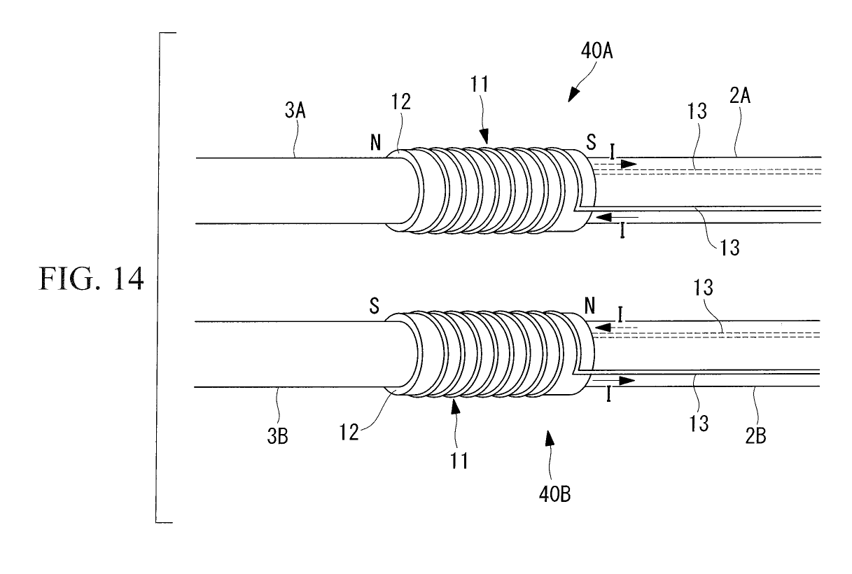

In this embodiment, as shown in FIG. 13 and FIG. 14, electromagnets 4A′ and 4B′ having coils 11 may be used, instead of the permanent magnets 4A and 4B. In this modification, the magnitude of the magnetic force of the electromagnets 4A′ and 4B′ can be changed by adjusting the current supplied to the coils 11 from a power supply (manipulation part) (not shown) provided at the proximal end side of the insertion sections 2A and 2B. Accordingly, coupling and uncoupling of the fixing parts 40A and 40B can be easily manipulated by adjusting the currents supplied to the coils 11.

The configuration of the electromagnets 4A′ and 4B′ may be selected as appropriate. For example, as shown in FIG. 13, the coils 11 may be formed by winding coated electric wires a plurality of times in grooves provided in the outer circumferential surfaces of the tube members 8. Alternatively, as shown in FIG. 14, the coils 11 may be solenoids formed by helically winding coated electric wires on the outer circumferential surfaces of the insertion sections 2A and 2B. In this case, the magnetic polarity of the two longitudinal ends of the solenoids 11 is determined according to the direction of currents flowing through the solenoids 11. Hence, by making the directions of currents I flowing through the solenoids 11 provided on the two insertion sections 2A and 2B be opposite to each other, one solenoid 11 has an N-pole distal end and an S-pole proximal end, and the other solenoid 11 has an S-pole distal end and an N-pole other end. Thus, the same advantage as that achieved with the fixing parts 4A, 4B, 4C, and 4D, as shown in FIG. 11, can be obtained.

It is desirable that core members 12 made of a ferromagnetic material, such as iron, be provided inside the coils 11 to increase the magnetic force of the electromagnets 4A′ and 4B′.

Electric wires 13 for supplying currents from the power supply to the coils 11 may be printed wires formed on the side walls of the insertion sections 2A and 2B. This enables the electric wires 13 to be formed as part of the insertion sections 2A and 2B.

In this embodiment, although the fixing parts provided on the two insertion sections 2A and 2B have the magnets 4A and 4B and the magnets 4A′ and 4B′ having different magnetic polarities, instead, one fixing part may have magnets, and the other fixing part may have temporary magnets that generate magnetic force by magnetic fields generated by the magnets.

In this embodiment, the fixing parts may be of a type other than magnets, as long as they can be coupled and separated by the manipulation at the proximal end portions of the sheaths 1A and 1B. For example, the fixing parts may be hook and loop fasteners that are fixed to the outer circumferential surfaces of the insertion sections 2A and 2B and that are coupled together by contact. Alternatively, the fixing parts may include a dovetail groove formed in one of the insertion sections 2A and 2B and a dovetail projection formed in the other, and the insertion sections 2A and 2B may be coupled together by fitting the dovetail projection into the dovetail groove.

In this embodiment, although the medical system 100 includes the two sheaths 1A and 1B, it may include three or more sheaths.

In this embodiment, although the sheaths 1A and 1B that are used inside the pericardial cavity Y have been shown as an example of the medical instrument, the fixing parts 4A, 4B, 40A, and 40B described in this embodiment may be directly provided on the endoscope 20 and the treatment instrument 30, or they may be provided on an introducer or a catheter used in a blood vessel.

As a result, the following aspect is read by the above described embodiment of the present invention.

To achieve the above-described object, the present invention provides the following solutions.

An aspect of the present invention provides a medical system including: a plurality of medical instruments each having an elongated insertion section that can be inserted into the body from the body surface; fixing parts that are provided on the insertion sections of the plurality of medical instruments, at positions away from the distal ends toward the proximal end side, and that can be coupled with and separated from each other; and manipulation parts that are provided at the proximal end side of the insertion sections of the plurality of medical instruments and that manipulate coupling and separation of the fixing parts.

According to this aspect, the insertion sections of the plurality of medical instruments are inserted into the same body cavity, and the fixing parts are coupled together inside the body cavity by manipulating the manipulation parts located on the outside of the body. This makes it possible to maintain the relative positions of the distal end portions of the plurality of insertion sections substantially constant and to integrally manipulate the distal end portions of the plurality of insertion sections inside the body cavity. Hence, the plurality of medical instruments can be simultaneously and easily manipulated even in a place like the pericardial cavity, where the medical instruments directly receive body movements.

In the above aspect, the fixing parts may include magnetic bodies generating magnetic forces that attract each other.

This allows the fixing parts to be spontaneously coupled together by magnetic force simply by bringing the fixing parts toward each other.

In the above aspect, the magnetic bodies may be electromagnets.

This makes it possible to adjust the coupling force between the fixing parts and, thus, to easily manipulate coupling and separation of the fixing parts.

In the above aspect, at least one of the medical instruments may include a flexible bending section provided closer to the distal-end side than the fixing part is.

This makes it possible to move the distal ends of the insertion sections by bending movement of the bending sections, even in a state in which the insertion sections are coupled together at the fixing parts.

In the above aspect, the medical system may further include a position changing mechanism that changes the positions of the fixing parts in the longitudinal direction of the insertion sections.

This makes it possible to change the positions of the fixing parts such that the plurality of insertion sections are coupled together at positions suited for the situation.

In the above aspect, the medical system may further include an anti-rotation mechanism that prevents the plurality of insertion sections coupled with each other at the fixing parts from relatively rotating about an axis extending in a direction passing through the fixing parts and intersecting the direction in which the plurality of insertion sections are arranged.

This makes it possible to stably maintain side-by-side arrangement of the distal end portions of the plurality of insertion sections that are coupled together at the fixing parts.

In the above aspect, the anti-rotation mechanism may include the plurality fixing parts provided on each of the plurality of insertion sections, at positions away from each other in the longitudinal direction. Alternatively, the anti-rotation mechanisms may include steps that are formed at contact surfaces of the fixing parts and that are engaged with each other in the radial direction of the insertion sections.

This makes it possible to effectively inhibit relative rotation of the distal end portions of the insertion section with a simple configuration.

In the above aspect, the medical instrument may be an endoscope, a sheath, or an introducer.

### REFERENCE SIGNS LIST

100 medical system
1A and 1B sheath
2A and 2B insertion section
2a and 2b lumen
3A and 3B bending section
4A, 4B, 4C, and 4D fixing part, permanent magnet
4A′ and 4B′ electromagnet
40A and 40B fixing part
5A and 5B manipulation part
6 over tube
7 flat surface
8 tube member (position changing mechanism)
9 manipulation wire (position changing mechanism)
10 step
11 coil, solenoid
12 core member
13 electric wire
20 endoscope
30 treatment instrument

1. A medical system comprising:
a plurality of medical instruments each having an elongated insertion section that can be inserted into the body from the body surface;
fixing parts that are provided on the insertion sections of the plurality of medical instruments, at positions away from the distal ends toward the proximal end side, and that can be coupled with and separated from each other; and
manipulation parts that are provided at the proximal end side of the insertion sections of the plurality of medical instruments and that manipulate coupling and separation of the fixing parts.

2. The medical system according to claim 1, wherein the fixing parts include magnetic bodies generating magnetic forces that attract each other.

3. The medical system according to claim 2, wherein the magnetic bodies are electromagnets.

4. The medical system according to claim 1, wherein at least one of the medical instruments includes a flexible bending section provided closer to the distal-end side than the fixing part is.

5. The medical system according to claim 1, further comprising a position changing mechanism that changes the positions of the fixing parts in the longitudinal direction of the insertion sections.

6. The medical system according to claim 1, further comprising an anti-rotation mechanism that prevents the plurality of insertion sections coupled with each other at the fixing parts from relatively rotating about an axis extending in a direction passing through the fixing parts and intersecting the direction in which the plurality of insertion sections are arranged.

7. The medical system according to claim 6, wherein the anti-rotation mechanism includes the plurality of fixing parts provided on each of the plurality of insertion sections, at positions away from each other in the longitudinal direction.

8. The medical system according to claim 6, wherein the anti-rotation mechanism includes steps that are formed at contact surfaces of the fixing parts and that are engaged with each other in the radial direction of the insertion sections.

9. The medical system according to claim 1, wherein the medical instrument is an endoscope, a sheath, or an introducer.
